(19) United States
(12) Patent Application Publication
(10) Pub. No.: US 2002/0121273 A1
(43) Pub. Date: Sep. 5, 2002

(54) Breast cancer treatment support bra

(76) Inventor: Cindy Nyilas, London (CA)

Correspondence Address: CINDY NYILAS, 12 HOPE ST., LONDON, ON, N6B 2C2, CA

(21) Appl. No.: 10/020,254

(22) Filed: Dec. 14, 2001

(30) Foreign Application Priority Data
Jan. 4, 2001 (CA) 2 327 753

(51) Int. Cl. A61F 5/28

(52) U.S. Cl. 128/99.1

(57) ABSTRACT

The present invention is a support bra for use by women after breast surgery. It is specifically designed to promote healing and reduce the chance of infection for women who have had a lumpectomy and are undergoing radiation treatment for breast cancer. The bra, consisting of two elongated bodice panels attached to three shaped cup panels, features external seams for greater comfort against the skin. The design allows women with limited mobility to easily put the bra on independently.

### FIELD OF THE INVENTION

The present invention relates to breast support for women who have been diagnosed with breast cancer and are choosing a lumpectomy and radiotherapy as the method of treatment and for all women going through breast cancer treatment who remain two-breasted.

### BACKGROUND OF INVENTION

Following the diagnosis of breast cancer, a woman and her physician must decide which kind of treatment she will undergo to fight the disease. There are generally two treatment types available for women in the early stages of breast cancer: mastectomy or lumpectomy. A mastectomy is a surgical procedure that removes the entire breast, while a lumpectomy is a surgical procedure that removes only the diseased part of the breast plus some of the surrounding tissue, leaving much of the breast intact. Until the mid 1980's, the standard treatment for breast cancer was a mastectomy. Since then, evidence has shown that a lumpectomy followed by radiotherapy is as effective as a full mastectomy. Many women are now opting to pursue this method of treatment.

After surgery, a woman's breasts need to be properly supported so as not to stretch out the incisions. Many women have sutures located directly under the breasts which is where most conventional bras hug the body. This can cause irritation and infection to the incision area. The entire breast and surrounding area can also suffer burns from the radiation treatment. Also, most women who have been diagnosed with breast cancer will have an axillary dissection This is a surgical procedure that removes lymph nodes from under the arm to see if the cancer has spread. Complications from this procedure are relatively frequent. Some of the complications include numbness in the arm and pain in the arm pit or down the arm. Some patients suffer from lymphedema which is the pooling of lymph fluids in the arm. The result of this condition is swelling and stiffness in the arm and shoulder. Invasive surgery and follow up treatment can result in a woman losing mobility on the affected side of her body. A woman's body is put through a great deal of stress during the diagnosis and treatment of breast cancer. Women who have been diagnosed with breast cancer and who have decided to have a lumpectomy followed by radiation have very special needs.

There are many undergarments and prosthetics available for women who lose an entire breast to cancer. Mastectomy bras coupled with prosthetics, are designed to help a woman maintain a natural appearance following the loss of a breast. These undergarment alternatives are not suitable for two-breasted women undergoing treatment for breast cancer. A woman who has had a lumpectomy and who is going through radiation treatment does not need a bra to camouflage what is missing, but rather needs a bra that cares for and supports the injured breast and the healthy breast.

It is currently recommended that women wear a sports bra during treatment. Sports bras are designed to hold a woman's breast firmly in place during physical activity. They typically have no fasteners and are put on over the wearer's head, which can be quite difficult and painful for a woman who has just had breast surgery. They are typically held in place by a thick elastic positioned around the wearer's body just under the breast. This elastic goes completely around the front and back of the body and is quite snug to stop the bra from riding up the wearer's chest. The area just under the woman's breast (called the fold area), is a major location for infection. The standard sports bra could cause unnecessary infection for a woman during the treatment of breast cancer because these bras are not taking her special needs into consideration.

It is important for comfort, and in the healing process, to cradle a woman's breasts without excessive compression, to gently lift the breasts at the fold area without any component of the bra digging in at this sensitive area. It is essential to provide good breathe-ability and comfort to the breasts, under the breasts at the fold, and to the underarm area. It is also important to consider the mental well-being of someone undergoing treatment for cancer and to provide them with the ability to get dressed and undressed independently.

### SUMMARY OF THE INVENTION

The invention is a garment to be worn by women undergoing treatment for breast cancer. More specifically it is to be worn by two-breasted women who choose a lumpectomy followed by radiation or a mastectomy followed by breast reconstruction instead of a mastectomy as a means of treatment.

The breast cancer treatment support bra is made from a stretchy and breathable fabric. It covers a woman's breasts, holding them gently in place without binding any injured areas. The elongated bodice section of the undergarment ensures that the garment will not finish on an area that has been sutured. The wide U-shaped, under the bust, elasticized seam cradles the breasts and elevates them so that their natural weight does not pull down and stretch out sutures. The bra can easily be put on by a woman with limited mobility and fastened via the diagonal detachable zipper located opposite the injured side of the body, using the non-injured arm.

The garment is designed and constructed to assist in the healing process by helping to control and minimize moisture traps, lifting the breasts at the fold area and keeping the breasts well supported. The garment can also assist in holding dressings in place and therefore help to eliminate the need for tape. All shaping seams are directed away from the body to create a smooth internal finish making the garment very comfortable against the skin.

### BRIEF DESCRIPTION OF THE DRAWINGS

FIG. 1 is a front view of the bra.

FIG. 2 is a side-front view of the bra.

FIG. 3 is a back view of the bra.

FIG. 4 is a side-back view of the bra.

FIG. 5 is a non-injured side view of the bra.

FIG. 6 is an injured side view of the bra.

FIG. 7 is a side-front view of the bra with the zipper opened.

FIG. 8 is a side-front view of the bra with the zipper opened illustrating the zipper facing.

FIG. 9 is a side-front view of the wearer putting on the bra.

FIG. 10 is a four sided view of a modification of the bra with an inserted sleeve.

FIG. 11 is a four sided view of a modification of the bra without an under the bust seam.

FIG. 12 is a four sided view of a modification of the bra without an under the bust seam and with inserted sleeves.

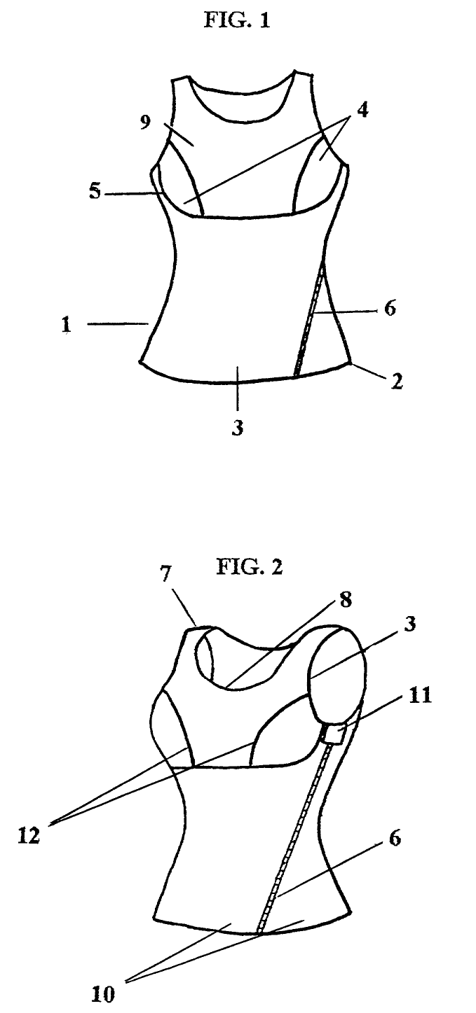

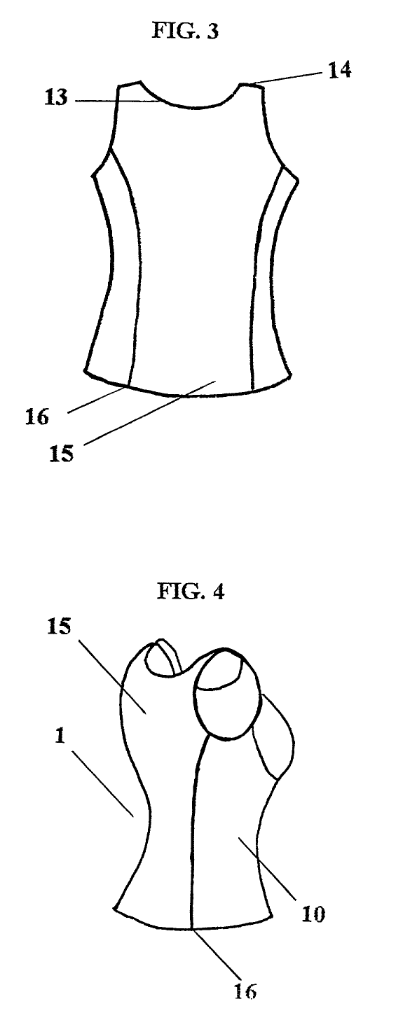

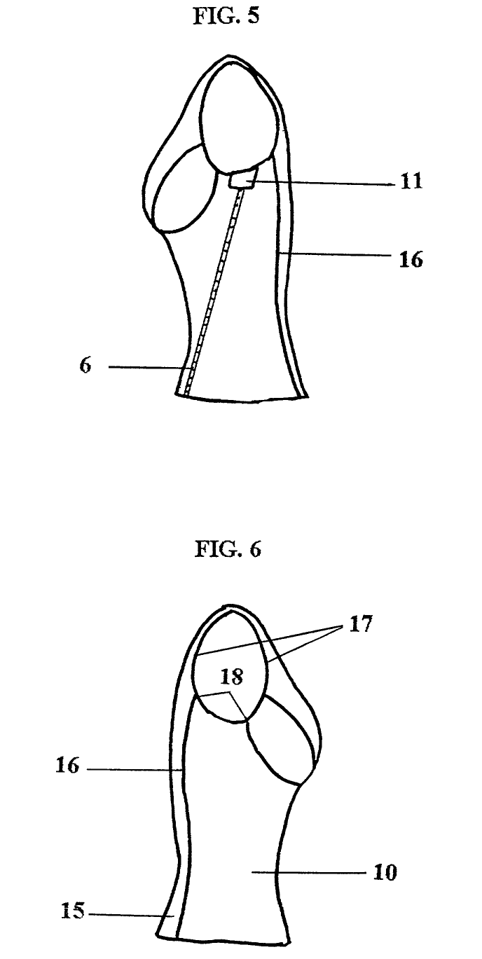

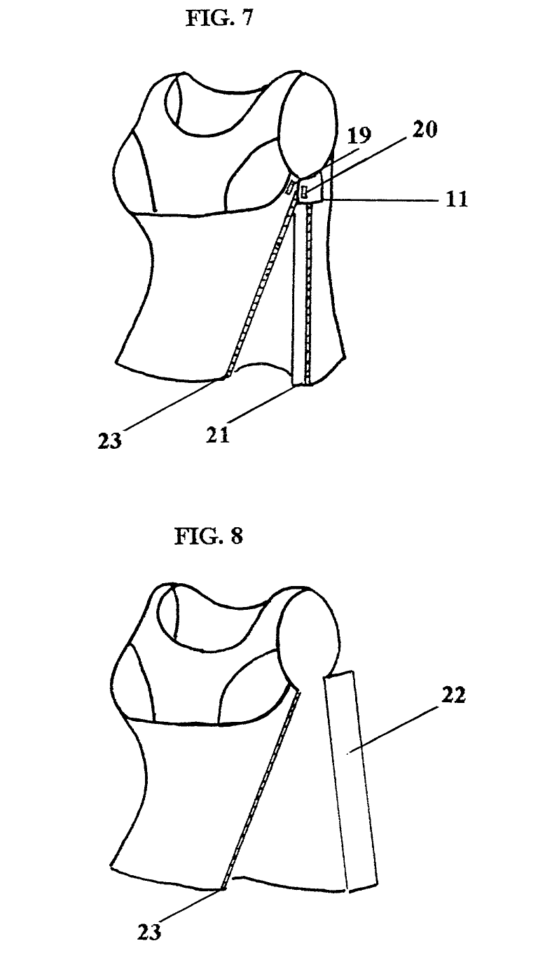

### DETAILED DESCRIPTION OF THE INVENTION

This support bra is designed specifically for women undergoing treatment for breast cancer. This bra has been invented to support a woman's breasts after surgery and during treatment. It is to be worn by two-breasted women after having a lumpectomy and during radiation therapy and for women who have breast reconstruction following a mastectomy. The comfort garment can also be worn by women undergoing any type of breast surgery where both breasts need to be supported during the healing process.

During the treatment of breast cancer, the breasts and surrounding area are very prone to irritation and infection. The bra has an elongated silhouette (1) that extends down to the hip area (2). The long bodice of the bra ensures that the garment will not finish at a sensitive area, therefore reducing the chance of complications by reducing the amount of irritation. The elongated bodice panels can also be tucked into the wearer's panties to help keep the garment in place.

The bra comprises of two bodice panels (10 & 15). These two panels are attached together by two vertical curvilinear seams located at the wearer's back. They are positioned approximately 4 to 7 inches from the center of the back of the garment (approximately 8 to 14 inches away from each other). The bodice shaping seams, like all of the shaping seams on the bra, are sewn together with the seams facing the outside of the garment. These external seams are then sewn down with a cover stitch or taping (again on the outside of the garment), making the seams on the outside of the bra reasonably thin and flat. The interior of the garment is constructed to be as smooth as possible for greater comfort against the wearer's skin. The interior smoothness allows for shaping and support without aggravating injured areas. The cups are comprised of three panels, the center front panel (9) and two side panels (4) are sewn together to form and support the wearer's breasts. The sewing of the front panels is the same as the bodice seams (external seams) (12). The cup section is fully lined (4&9) for greater stability. The front shoulders of the cup section are approximately two to three inches wide (7). The back shoulders of the corresponding bodice panels are also two to three inches wide (14). The generous width given to the shoulder area of the garment allows for even breast weight distribution on the wearer's shoulders. This translates into greater comfort to the wearer by alleviating any pressure points on the wearer's shoulders. The front neck of the cup section is located one to three inches below the wearer's collarbone (8) in order to cover, but not to end on, any area of the wearer's chest that may be sutured or burned by radiation. The back neckline (13) of the garment is three to five inches below the nape of the neck. The high back neckline of the garment helps to stabilize the back shoulders of the garment as well as support the back armholes. On the front section of the bodice panel, there is a wide U-shaped seam (5) to accommodate the three cup panels (4 & 9). The bottom section of the three front shaped cup panels is attached to the wide U-shaped seam of the bodice panels. An elastic measuring ¼ to 1 inch in width is inserted into this seam (5). The elastic is sewn on the outside of the garment so as not to touch the wearer's skin. After being attached to the exterior of the wide U-shaped seam, the elastic is then sewn down and held in place with a cover stitch or taping which is once again on the outside of the garment, leaving the inside of the garment smooth and flat against the wearer's skin.

The front and back neckline (8&13) have ¼ to ½ inch elastic sewn onto the edges of the fabric. The sewn-on elastic is then turned over towards the inside of the garment and sewn down using a cover stitch. This elastic is in place to give the front and back neckline good stability and integrity, to keep the neckline sections of the bra in place and to ensure that the neckline returns to the same shape and fit after repetitive wearing and laundering. The armholes of the bra have ¼ to ½ inch elastic sewn in (17) from the wide, U-shaped, under the cup seam (5) up towards the shoulder, all the way to the back bodice shaping seam (16). Continuing along the armhole, from the back bodice-shaping seam across the armpit area and back up to the wide U-shaped seam (5), there is less tension in the elastic inserted (18). Along the entire armhole the fabric is sewn down by the same width as the elastic. This includes the armpit area which has less elastic tension (19). The bottom section of the armhole has less elastic tension for greater comfort to the wearer.

Anyone who has been diagnosed with breast cancer will likely have to undergo a surgical procedure called an axillary dissection. During this procedure, lymph nodes are removed from a patient's armpit area to assess whether the cancer has spread. Following this surgery, many women suffer from complications which can include pain, swelling, numbness and stiffness of the arm or armpit. It is because of these frequent complications that the underarm of the bra is constructed to be as unobtrusive as possible. The stability and strength of the entire armhole is put in place by the greater elastic tension on the top section only (17).

From the initial discovery of a lump or abnormality in a breast to a diagnosis and treatment, a woman's body and mind is put through a great deal of stress. Because of this physical strain, sometimes mobility can become limited. On the diseased side of a woman's body (25), many invasive surgical procedures as well as radiation treatments may be necessary. This can cause damage to muscle tissue, nerves and the skin. The result can be a temporary lack of mobility to that side of the body. The bra is very easy to put on for a woman with a limited range of motion on one side of her body. During such difficult emotional times, the independence associated with being able to dress one's self can be of great emotional benefit to the wearer. For this reason, the support bra is secured on the wearer's body by a detachable zipper (6). This zipper can be located on the left or right side of the garment, and is always on the non-injured side of the woman's body. The zipper begins approximately 3 to 6 inches from the center front of the bottom of the garment (23) (measuring from the center of the front, at the hem, horizontally towards the non-injured side of the body). It is at this point that the two sides of the zipper come together and are hooked in order to raise the pulley and do the zipper up, or unhooked in order to detach the zipper. The zipper angles from this point diagonally towards the armpit of the noninjured side of the body (24). Under the zipper, there is a 2 inch facing sewn to the side of the zipper closest to the back (22). The facing is positioned ⅜ of an inch further towards the back than the zipper. The rest of the facing sticks out from under the back half of the zipper, facing the front (21). This 2 inch rectangular fabric piece (22) is made of a soft fabric and acts as a physical barrier between the zipper and the wearer's skin. At the underarm level of the zipper, the fabric facing folds over the top section of the zipper towards the outside of garment (11). This 1½ to 2 inch fold-over section of the facing is to cover the zipper pulley when the bra is secured on the wearer's body in order to provide greater comfort in the armpit area of the woman's non-injured side. On the back half of this fold-over section, the facing is sewn down to the body of the garment. On the front half, the fold-over section of the facing is secured in place, when the zipper is fully done up, by corresponding pieces of hook and loop fastening material (VELCRO TM). Directly beside the top of the zipper towards the front, a small rectangular shaped piece of female VELCRO TM (loop) (19) is sewn onto the outside of the bra. On the underside of the front half of the fold-over section of the facing, the male side of the VELCRO TM (hook) (20) is sewn.

The support bra is to be constructed from a stretchy fabric with good breathe-ability. The preferred fabric is 80-90% COOLMAX TM and 10-20% LYCRA TM jersey knit. The knitted fabric will consist of four-way stretch and also have good fabric memory (does not lose it's shape). The front (10) and back (15) bodice panels are cut and sewn using a single layer of fabric whereas the three shaped cup panels (4&9) are cut and sewn using double layers of fabric for added stability. The main function of the preferred fabric is to wick moisture away from the body keeping the wearer dry and comfortable. The fabric is also very durable and can withstand frequent laundering and extended periods of wear without losing it's shape and fit. COOLMAX TM is a 100% polyester fiber that has proven wicking and fast drying capabilities, all while providing comfort, durability and case of care. The second fiber in the preferred fabric is LYCRA TM. This fiber offers great stretch and the ability to return to its original shape after being stretched out. Garments made with LYCRA TM in their fabric content stretch easily and consistently over the curves of the body, returning to their original shape.

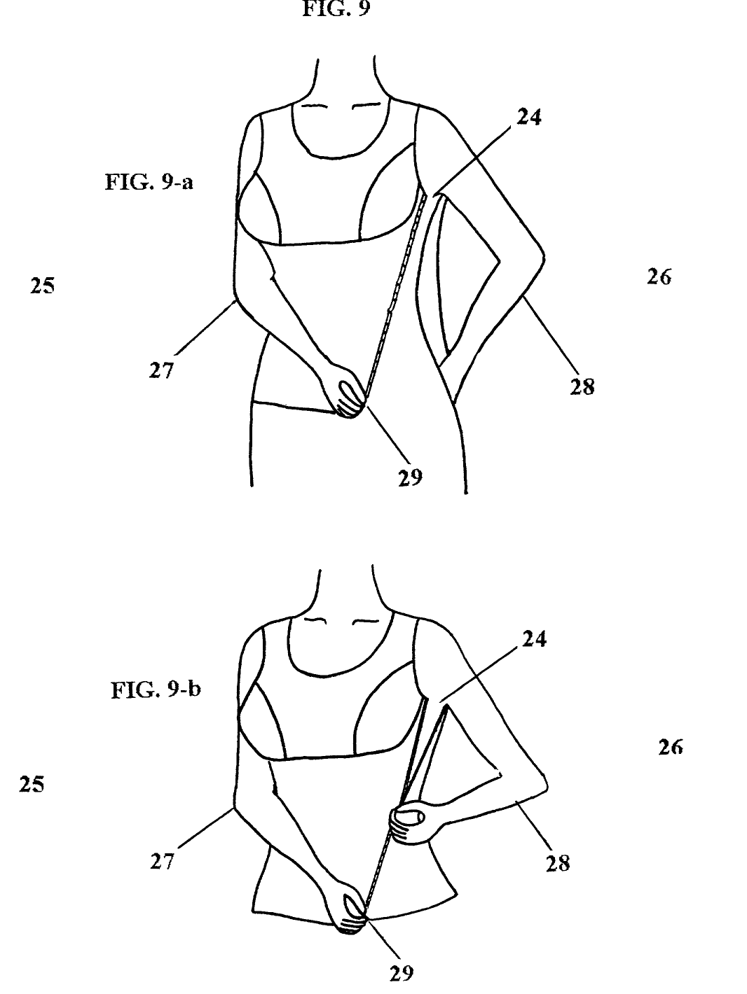

The bra is very easy to put on using one's non-injured side almost exclusively. A woman suffering from pain, discomfort, numbness or stiffness following axillary or breast surgery may not have full mobility of the arm or shoulder on the injured side of her body (25). When a decrease of mobility occurs, a woman will typically have her injured arm resting flat against the side of the body, her elbow bent at approximately 90 degrees and the lower half of her arm resting against her stomach if standing or against her stomach and on her lap if sitting (arm forming an L shape) (27). The bra can be completely secured on the wearer's body, by the wearer, without having to move the arm of the injured side from it's comfortable L-position. By holding the injured side shoulder of the bra with the non-injured side hand, one can easily slip the hand of the injured side into the armhole of the bra (moving the non-injured side only). Still using the non-injured arm (28), the bra can be lifted up the injured arm, and the injured side shoulder of the bra positioned on the injured side shoulder of the body. The wearer can then grab the non-injured side shoulder of the bra with her non-injured side hand to lift it over the head in order to place the non-injured shoulder of the bra on the non-injured side shoulder of the body. At this point the bra is completely on the wearer's body and needs only to be zipped up (FIG. 9a). To fasten the zipper, the wearer can hold the front half of the zipper at the bottom/beginning of the zipper using the thumb and forefinger of the hand located on the injured side of the body (29). Because the injured arm is naturally resting in an L position (27), the wearer does not have to reposition her arm or her hand to grip the bottom of the zipper. The only movement necessary to hold the beginning of the zipper (23) is from the thumb and forefinger (29) of the injured side. The zipper can be hooked and the pulley raised towards the armpit of the non-injured side. All the movement being done to fasten the zipper is accomplished by the non-injured side (26). Once the zipper is fastened, the wearer can then attach the VELCRO TM tabs (19&20) of the fold-over comfort facing to protect the skin from touching the zipper pulley. This is done using the non-injured side of the body (26). Any breast adjustments are then done using the non-injured arm and hand (28).

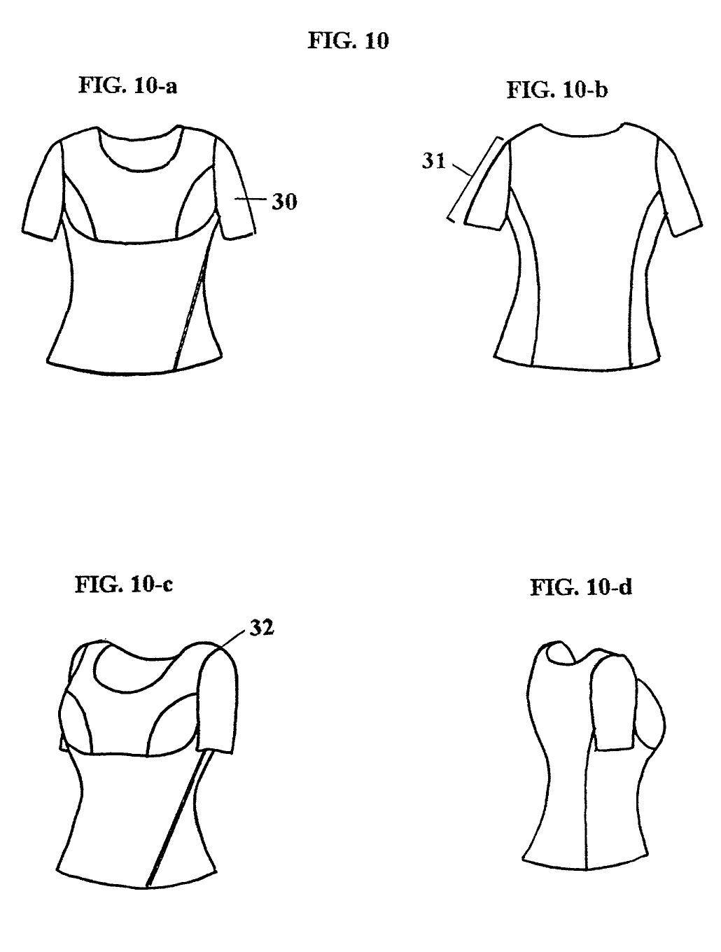

The comfort bra for women having surgery and who are undergoing treatment for breast cancer could also have several style variations. The basic style could have sleeves inserted in order to assist in keeping underarm dressings in place without the use of tape (FIG. 10). The fabric in the sleeves would also help to keep the wearer's armpits dry to lesson the chance of infection. The set-in sleeves are well fitted around the wearer's shoulders and underarms. The sleeves would be 7 to 10 inches long (31) (measuring from the shoulder where the sleeve meets the shoulder seam of the main body of the bra). The sleeves would be sewn in (32) with all seams facing the outside of the bra for greater comfort to the wearer. These seams would then be sewn down using a cover stitch or taping.

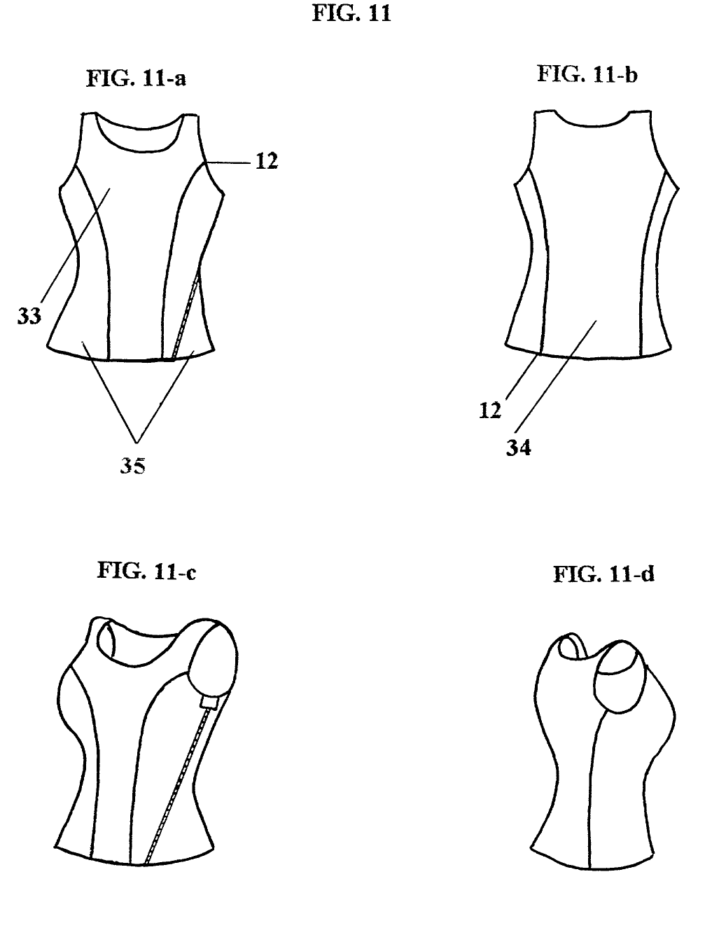

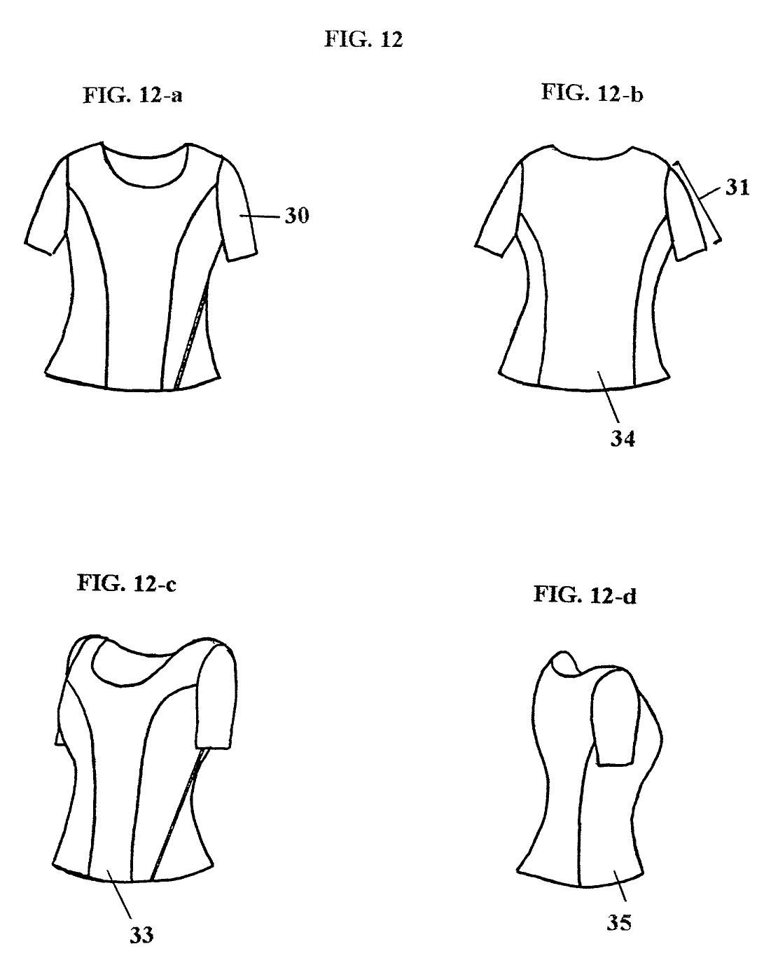

The bra could also be made without a wide, under the bust, U-shaped seam (FIG. 11). This style would be for smaller women who need some support but who do not need to have their breasts elevated drastically from their natural position. This variation would have two side panels (35) attached to a single front (33) and a single back panel (34) using external seams. (12) This style could also have sleeves inserted for women who need dressings to be held in place in the underarm area or for women who need to keep the armpit area dry in order to avoid infection (FIG. 12). The front and back shaping seams as well as the armhole seams of the sleeves are all sewn using external seams for greater comfort to the wearer.

What is claimed is:

1. A breast cancer treatment support bra for two breasted women undergoing treatment for breast cancer comprising:
a front and a back panel each of which extends to the wearer's hips, said front panel includes a U-shaped upper periphery, two vertical curvilinear edges and a lower edge, said back panel includes two upper flat shoulder edges joined by a curved edge neckline at the upper end, two vertical curvilinear edges matching said vertical curvilinear edges of the said front panel and a lower edge, the said vertical curvilinear edges of both the front and back panels are joined by a shaped seam to form a bodice, cups with a U-shaped lower periphery matching the u-shaped upper periphery of said front panel of said bodice, said cups comprised of a center front panel and two side panels, said center front panel including an upper curved neckline joined on each side by two upper flat shoulder edges with said shoulder edges merging into curved armhole edges which extend vertically downwardly whereupon they connect with the arc shaped edges whose distal ends terminate at the extremity of said U-shaped lower edge; said cups comprising two side panels, said side panels each include an inner arc shaped edge, each of said arc shaped edges join the two curvilinear arc shaped edges of the center front panel by a shaped seam to create the cups; said two side panels include a outer arc shaped edge that is attached to the upper u-shaped periphery of the front panel of the bodice; at the outer extremity of said u-shaped seam, said side panels merge upwardly to create part of the armhole edges; said bodice and cups are joined together at said u-shaped peripheries by an elasticized seam, said two upper flat shoulder edges of the back panel and said two upper flat shoulder edges of the center front panel are attached to create a breast cancer treatment support bra, said bra being adapted for detachable assembly on a wearer using a diagonally placed detachable zipper located on the front panel, said diagonally placed detachable zipper assembly is positioned from the lower edge of the front panel and extends upwardly and diagonally to terminate at the edge of the front panel armhole, said diagonally placed detachable zipper assembly can be positioned on the left or right side of the bra to accommodate the wearer.

2. A breast cancer treatment support bra as claimed in claim 1, wherein said curvilinear shaped seams that attach to form the bodice are located on the back of the wearer's body to avoid contact with the injured areas of the wearer.

3. A breast cancer treatment support bra as claimed in claim 1, wherein all shaped seams are sewn towards the outside of the bra for greater comfort against the skin, and all external shaping scams are covered with a cover stitch or taping for smoothness on the outside of the bra.

4. A breast cancer treatment support bra as claimed in claim 1, wherein there is a ¼ to ½ inch elastic around the rounded front and back necklines and around the armholes of the bra.

5. A breast cancer treatment support bra as claimed in claim 1, wherein said bra can be adapted for detachable assembly on a wearer using a diagonally placed detachable zipper located on the front panel; said detachable zipper begins at the horizontal lower edge of the front panel at the hip level of the wearer ½ way between the center front to the side of the wearer's body.

6. A breast cancer treatment support bra as claimed in claim 5, wherein said detachable zipper angles diagonally towards the underarm of the non-injured side of the wearer and can be fastened by a wearer with limited mobility on the injured side of the wearer using the non-injured side hand and arm.

7. A breast cancer treatment support bra as claimed in claim 5, wherein said bra can be fastened and unfastened by a wearer requiring minimal movement from the injured side of the wearer's body.

8. A breast cancer treatment support bra as claimed in claim 5, wherein under said detachable zipper assembly there is a 2 inch soft fabric facing attached to the inside of the bra to provide a barrier between the detachable zipper and the skin of the wearer for greater comfort to the wearer, said facing folding 1½ to 2 inches over the top of the outside of the garment for greater comfort at the underarm level of the wearer.

9. A breast cancer treatment support bra as claimed in claim 8, wherein said fold-over section of the facing is sewn down at the underarm level of the breast cancer treatment support bra on the back half of the zipper assembly and fastened by VELCRO TM on the front half of the zipper assembly.

10. A breast cancer treatment support bra as claimed in claim 1, wherein said breast cancer treatment support bra is made from stretchy fabric with good breath-ability, preferably 80 to 90% COOLMAX TM and 10 to 20% LYCRA TM knit fabric.

11. A breast cancer treatment support bra as claimed in claim 1, wherein said bra could have sleeves to hold underarm dressings in place on the wearer.

12. A breast cancer treatment support bra as claimed in claim 1, wherein said breast cancer treatment support bra could be constructed without the U-shaped elasticized seam and with the curvilinear seams attaching the center front panel to the two side panels to form the cups extending down vertically to the lower extremity of the front panel of the bodice to the hip level of the wearer.

13. A breast cancer treatment support bra as claimed in claim 1, wherein the sizing of said bra is determined by bodice size and cup size independently of each other.

14. A breast cancer treatment support bra as claimed in claim 1, wherein said bra is designed to hold breasts in place so that sutures can heal without being stretched out by the natural weight of a wearer's breasts, to hold dressings in place, to comfortably support the breast's of the wearer while radiation burns heal, to lift and support breasts without excessive compression, to cover but not bind injured areas, to provide a physical barrier under the wearer's breasts at the fold area to reduce the chance of infection and irritation caused by skin on skin friction and moisture traps, to keep all areas of the breasts dry and properly aerated, and to allow a wearer with limited mobility to put the bra on independently.
